(19) United States
(12) Patent Application Publication
(10) Pub. No.: US 2008/0286147 A1
(43) Pub. Date: Nov. 20, 2008

(54) Apparatus and Method for Decontamination

(75) Inventors: Deborah E. Wilson, Dunn Loring, VA (US); Murray L. Cohen, Fort Worth, TX (US); Thomas McWhorter, Allentown, PA (US)

Correspondence Address: KLARQUIST SPARKMAN, LLP, 121 S.W. SALMON STREET, SUITE #1600, PORTLAND, OR, 97204-2988, US

(73) Assignees: GOVERNMENT OF THE U.S.A. OF AMERICA AS REPRESENTED, Rockville, MD (US); CDG RESEARCH CORPORATION, Bethlehem, PA (US); CDIC, INC., Atlanta, GA (US)

(21) Appl. No.: 11/666,054

(22) PCT Filed: Oct. 18, 2005
(86) PCT No.: PCT/US05/37753

§ 371 Date: Apr. 18, 2007

Related U.S. Application Data
(60) Provisional application No. 60/620,095, filed on Oct. 18, 2004.

(51) Int. Cl. A61L 2/20

(52) U.S. Cl. 422/37; 422/292

(57) ABSTRACT

The present disclosure relates to the decontamination of articles or areas contaminated with one or more pathogens. Methods and apparatuses are disclosed that can be used to decontaminate an environment, for example a room, building, or articles contaminated with a pathogen.

### CROSS-REFERENCE TO RELATED APPLICATION

This application claims the benefit of U.S. Provisional Application No. 60/620,095 filed Oct. 18, 2004, herein incorporated by reference.

### FIELD

The present disclosure relates to the decontamination of articles and areas that are or may be contaminated with pathogens.

### BACKGROUND

There is a great need for effective methods and devices for neutralizing pathogens, such as biosafety level 2, 3 and 4 agents. For example, many laboratories throughout the world perform research on pathogens, resulting in contaminated equipment and research laboratories. In many instances, contaminated equipment is destroyed, for example by incineration, due to the difficulties in cost-effectively neutralizing the pathogens.

While formaldehyde gas has been used as a decontaminant for over 100 years, the efficacy of this process remains controversial (Munro et al., Appl. Environ. Microbiol. 65:873-6, 1999). Decontamination using formaldehyde involves the depolymerization of paraformaldehyde to create a toxic formaldehyde gas. The formaldehyde gas is maintained in contact with the object to be decontaminated (such as inside a biological safety cabinet) for several hours (for example 4-6 hours, see Fink et al., Am. Ind. Hyg. Assoc. J. 49:277-9, 1988), which can result in the cabinet being unavailable for use for one or two days. In addition, because the formaldehyde gas generated is highly toxic and a suspected carcinogen, it is advisable for persons to leave the room during the decontamination process. This can result in significant downtime for a laboratory. After the desired period of contact with the object, the formaldehyde is neutralized with either ammonium carbonate or potassium permanganate and the cabinet is vented.

Another method that has been used to decontaminate biological safety cabinets is hydrogen peroxide (H₂O₂) vapor. For example, Steris Corporation uses Vaporized Hydrogen Peroxide (VHP®) to decontaminate anthrax-exposed buildings. Sabre Technical Services uses chlorine dioxide to decontaminate anthrax-exposed buildings. One advantage of chlorine dioxide is that it does not have the carcinogenicity, mutagenicity or teratogenicity that is ascribed to formaldehyde. However, current methods of using VHP® and chlorine dioxide require large amounts of equipment transported on a truck, and take several hours to complete the decontamination process. Such methods and devices are impractical in the context of decontaminating laboratory equipment or decontamination of an individual area (such as a laboratory) within a larger building.

Thus, there is a need for methods and portable equipment that can be used to neutralize pathogens quickly, without substantial adverse impact on the contaminated object or the environment.

### SUMMARY

Provided herein are apparatuses for decontaminating an environment, such as an article or an area (for example a room or building) contaminated or thought to be contaminated with pathogens, such as a biosafety level 2, 3, or 4 pathogen. In particular examples, the article to be decontaminated is a piece of laboratory equipment, such as a biological safety cabinet.

In particular examples, the apparatus is portable, and includes a moveable cart, a source of chlorine dioxide gas and a humidifier on the cart, an inlet conduit for introducing a flow of chlorine dioxide gas from the source of chlorine dioxide gas into the environment and for humidifying the environment, and an outlet conduit for withdrawing gas from the environment. The apparatus can further include a blower for circulating gas in the environment from the environment to the humidifier and from the humidifier to the environment. The moveable cart can be a wheeled cart, such as a cart with at least two wheels, at least three wheels, or at least four wheels. In particular examples the apparatus weighs less than 200 pounds, such as 50-200 pounds.

The inlet conduit can be a single unit through which the flow of chlorine dioxide gas and humidifier communicate with the environment. In another example, the inlet conduit includes a first unit through which the flow of chlorine dioxide gas communicates with the environment and a second unit through which humidifier communicates with the environment. The outlet conduit can be a single unit through which the environment communicates with the humidifier or blower.

The blower can include a blower inlet conduit and a blower outlet conduit that establishes a path of flow between blower inlet and blower outlet. The blower inlet is in fluid communication with the outlet conduit for withdrawing gas from the environment to establish a path of flow between the environment and the blower. The blower outlet is in fluid communication with a humidifier inlet conduit on the humidifier to establish a path of flow between the humidifier and the blower.

The humidifier can include a humidifier inlet conduit and a humidifier outlet conduit to establish a path of flow between humidifier inlet and humidifier outlet, wherein the humidifier inlet is in fluid communication with the blower outlet conduit to establish a path of flow between the humidifier and the blower. The humidifier outlet is in fluid communication with an inlet conduit for introducing a flow of chlorine dioxide gas into the environment to establish a path of flow between the humidifier and the environment.

The apparatus can further include a scrubber for neutralizing chlorine dioxide gas. In particular examples, the scrubber and the humidifier comprise a single unit. For example, the single unit can include a chamber for holding liquids. When humidification is desired, water is added to the chamber; when neutralization is desired, a chlorine dioxide neutralizing solution can be added to the chamber. In some examples, the blower is part of the scrubber/humidifier unit and permits air or other gases to be circulated through the scrubber/humidifier.

The source of chlorine dioxide gas can include an outlet conduit that is in fluid communication with the inlet conduit for introducing a flow of chlorine dioxide, thereby establishing a path of flow between source of chlorine dioxide gas and the environment.

In some examples, the apparatus also includes a diluted chlorine gas in a flow path with the source of chlorine dioxide gas, such as a chlorine dioxide gas generator. The source of diluted chlorine gas can include a diluted chlorine gas outlet conduit that is in fluid communication with a chlorine dioxide generator inlet conduit on the chlorine dioxide generator, thereby establishing a path of flow between the chlorine dioxide generator and the diluted chlorine gas.

The disclosed apparatuses can further include detection and monitoring devices, such as a hygrometer and a device for measuring chlorine dioxide concentration. In addition, any of the flow paths on the apparatus can include a flow regulator, rotometer, on/off valve, or combinations thereof to regulate the flow of gas.

The disclosed apparatuses can be used to decontaminate an environment containing or thought to contain a pathogenic spore. For example, the method can include sealing the environment, attaching the inlet conduit and outlet conduit of the apparatus to the environment, humidifying the environment, and introducing a flow of chlorine dioxide gas through the environment as described herein.

Also provided are decontamination methods for pathogens that permit decontamination of an environment, such as an article or an area. In some examples, the method uses a disclosed apparatus. In particular examples, the method includes providing a sealed environment, such as an environment that is substantially gas impermeable. For example, if the area to be decontaminated is a room or building, doors and windows are closed, and any cracks or openings to the outside sealed, to generate a sealed environment. If an article, such as a piece of laboratory equipment, is to be decontaminated, openings in the article are closed (for example with plastic sheeting and tape), to generate a sealed article. In particular examples, the decontamination can be completed in less than 3 hours, such as less than 2 hours, and even less than 1.5 hours. In particular examples, the method takes about 1-2 hours to complete, such as 1.5-2 hours to complete. For example, if the method includes humidification, introduction of gaseous chlorine dioxide, and neutralization, in particular examples all of these steps can be completed in less than 3 hours, such as less than 2 hours, for example less than 1.5 hours, for example 1-3 hours.

The sealed environment is humidified, for example to at least 70% relative humidity, and in some examples no more than 90% relative humidity. In some examples, the sealed environment is humidified for at least 10 minutes, such as at least 20 minutes, for example 10-20 minutes. In particular examples, humidification takes no longer than 30 minutes, such as no more than 20 minutes, for example 1-30 minutes. In particular examples in which the ambient humidity of the sealed environment is 70-90% relative humidity, no humidification is provided.

During humidification of the sealed environment, gaseous chlorine dioxide is introduced into the sealed environment. Ideally, the amount of chlorine dioxide added is effective to decontaminate the environment by killing substantially 100% of the pathogens present in the environment. In some examples, the chlorine dioxide gas in the sealed environment achieves a concentration of at least 1000 parts per million (ppm) chlorine dioxide, such as at least 2500 ppm or at least 4000 ppm chlorine dioxide. The gaseous chlorine dioxide can be continuously circulated through the sealed environment for at least one hour, such as for at least one hour but no more than two hours. In particular examples, the relative humidity of the sealed environment is at least 70% during the introduction of gaseous chlorine dioxide. In some examples, the sealed environment is humidified to at least 70% prior to introduction of the gaseous chlorine dioxide.

During the humidification and introduction of gaseous chlorine dioxide, humidified air or gaseous chlorine dioxide (or both) are continuously circulated through the sealed environment using a closed loop circulation system. For example, during humidification, humidified air is continuously circulated through the sealed environment using a closed loop circulation system. Humidified air is transported from the humidifier into the sealed environment via a fluid flow path, then out of the sealed environment back into the humidifier via another flow path. In some examples, humidified air is transported from the humidifier into the sealed environment via a fluid flow path, then out of the sealed environment through a blower, and back into the humidifier via another flow path. For example, the closed loop circulation system can include a source of humidified air (such as a humidifier) extended to the sealed environment, a gas inlet and a gas outlet that communicate with the sealed environment, and circulating the humidified air includes introducing the humidified air into the gas inlet and removing humidified air through the gas outlet.

Similarly, during decontamination, humidified air and gaseous chlorine dioxide are continuously circulated through the sealed environment using a closed loop circulation system. Chlorine dioxide gas is transported from the source of chlorine dioxide into the sealed environment via a fluid flow path, then out of the sealed environment back into the humidifier via another flow path. In some examples, chlorine dioxide gas is transported into the sealed environment via a fluid flow path, then out of the sealed environment into the humidifier via a flow path through a blower.

In particular examples, the closed loop circulation system of the present application includes a source of gaseous chlorine dioxide that communicates with the sealed environment. For example, a gas inlet and a gas outlet communicate with the sealed environment. Circulating the gaseous chorine dioxide includes introducing the gaseous chlorine dioxide under pressure into the gas inlet and removing gaseous chlorine dioxide through the gas outlet. In some examples, substantially equal volumes of gaseous chlorine dioxide are introduced into the gas inlet and removed through the gas outlet to promote a substantial steady-state of gaseous chlorine dioxide in the sealed environment.

Following the introduction of gaseous chlorine dioxide, the method can further include neutralizing the remaining chlorine dioxide gas in the sealed environment, for example using a neutralizing agent. In a particular example, after terminating the flow of chlorine dioxide gas from the source of chlorine dioxide, the gaseous chlorine dioxide in the sealed environment is transported to a solution that includes sodium thiosulfate hydrate and a sufficient amount of an inorganic base (such as NaOH) to maintain alkalinity of the solution during consumption of thiosulfate by the chorine dioxide. An exemplary neutralization solution is 10% by weight each sodium thiosulfate hydrate and NaOH in water. The gaseous chlorine dioxide in the sealed environment can be transported to the neutralization solution by circulating the gaseous chlorine dioxide between the sealed environment and the neutralization solution. In some examples, the neutralization is continued until the level of chlorine dioxide gas in the sealed environment is below 0.5 ppm, such as less than 0.1 ppm.

One or more of the humidification, introduction of gaseous chlorine dioxide, and neutralization steps can be performed in the absence of negative pressure, for example where the sealed environment is at ambient pressure during one or more of the steps. In particular examples, the sealed environment is at ambient pressure during humidification, introduction of gaseous chlorine dioxide, and neutralization.

In one example, the method includes providing a sealed environment, humidifying the sealed environment to at least 70% but not more than 90% relative humidity in absence of negative pressure, introducing into the sealed environment at least 1000 ppm gaseous chlorine dioxide (such as at least 2500 ppm or at least 4000 ppm gaseous chlorine dioxide) for at least one hour but no more than two hours in the absence of negative pressure, and neutralizing the gaseous chlorine dioxide in the sealed environment in absence of negative pressure. Humidified air is continually circulated through the sealed environment using a closed loop circulation system during humidification, introduction, and neutralization steps, and the gaseous chlorine dioxide is circulated through the sealed environment using a closed loop circulation system during the introduction, and neutralization steps.

In another example of the method, the method includes providing a sealed environment, humidifying the sealed environment to at least 70% relative humidity in absence of negative pressure, introducing into the sealed environment a concentration of gaseous chlorine dioxide effective to kill substantially 100% of pathogens present in the sealed environment, and continuously circulating air in the sealed environment through a humidifier during the humidification and introduction of gaseous chlorine dioxide steps. The method can further include neutralizing the gaseous chlorine dioxide in the sealed environment after continuously circulating the gaseous chlorine dioxide gas.

In some examples, the sealed environment is an inhabitable structure, such as a building. In such instances, the inhabitable structure can include a central or remote source of gaseous chlorine dioxide and a fluid flow path from the central source of gaseous chlorine dioxide to one or more rooms in the inhabitable structure. In another example, the source of gaseous chlorine dioxide is located outside of the inhabitable structure, and the fluid flow path runs from the source of gaseous chlorine dioxide into the inhabitable structure, such as into one or more areas of the structure. Introducing gaseous chlorine dioxide into areas of the inhabitable structure can therefore include introducing the gaseous chlorine dioxide through the fluid flow path into one or more areas. One or more rooms in the inhabitable structure can include an air circulation unit having a humidifier, and can further include a fan or blower, as well as a scrubber for neutralizing the gaseous chlorine dioxide.

In a particular example, a method of decontaminating an inhabitable structure is disclosed. The method can include sealing the inhabitable structure to substantially make the inhabitable structure gas-impermeable, to provide a sealed environment. The sealed environment is humidified to at least 70% relative humidity, but no more than 90% humidity in absence of negative pressure. However, if the sealed environment is already at 70%-90% relative humidity, the humidification step can be omitted if desired. During or after the humidification, at least 1000 ppm gaseous chlorine dioxide is introduced (such as at least 2500 ppm or at least 4000 ppm gaseous chlorine dioxide, for example 1000-4000 ppm gaseous chlorine dioxide) into the sealed environment for at least one hour but no more than two hours in absence of negative pressure in the sealed environment. Next, the gaseous chlorine dioxide in the sealed environment is neutralized in the absence of negative pressure in the sealed environment. Neutralizing can include exposing gaseous chlorine dioxide in the sealed environment to a solution including sodium thiosulfate and NaOH. Air or other gases in the sealed environment are continually circulated through a humidifier during the humidification and introduction of gaseous chlorine dioxide steps, and through a scrubber during the neutralization step.

Also disclosed are buildings, which are designed to be easily decontaminated. In one example, the building includes one or more laboratories, for example a laboratory contaminated or thought to be contaminated with a pathogen, such as a laboratory that routinely works with biosafety level 2, 3 or 4 pathogens. In particular examples, the building has multiple subunits, such as multiple rooms. In some examples, one or more of the multiple subunits are capable of being sealed so that the subunits are substantially gas-impermeable.

The building can include a central (for example a remote) source of chlorine dioxide gas that communicates with one or more of the multiple subunits through conduits, a humidifier that provides a source of humidified air, and a blower that circulates the chlorine dioxide gas and humidified air within one or more selected subunits of the building.

The central source of chlorine dioxide gas can be a room within the building, or outside of the building. Although the word “central” is used, this does not mean that that source of chlorine dioxide gas has to be in the center of the building, or in the building at all. The source of chlorine dioxide gas can be anywhere in the building or outside of the building. In addition, the building can include multiple sources of chlorine dioxide gas, for example one per floor, or one per side of the building. In examples where the central source of gaseous chlorine dioxide is located outside of the building, one or more fluid flow paths runs from one or more central sources of gaseous chlorine dioxide into the building.

In particular examples, the source of chlorine dioxide gas is a chlorine dioxide generator, and the central source of chlorine dioxide gas also includes a source of dilute chlorine gas in fluid communication with the chlorine dioxide generator. The connection between the dilute chlorine gas and the chlorine dioxide generator can include a fluid flow path, which can also include regulators, rotometers, on/off valves, and so on, for regulating the flow of the dilute chorine gas to the chlorine dioxide generator. Similarly, the fluid flow path from the source of chlorine dioxide gas can include regulators, rotometers, on/off valves, and so on, for regulating the flow of the chlorine dioxide gas to the areas within the building.

The disclosed buildings can also include a scrubber that provides a source of chlorine dioxide neutralizing agent to the selected subunits following decontamination with chlorine dioxide gas. The scrubber and humidifier can be separate units, or can be a single unit. For example, a single unit can include a chamber for holding liquids. When humidification is desired, water is added to the chamber; when neutralization is desired, a chlorine dioxide neutralizing solution can be added to the chamber. In some examples, a blower is provided as part of the scrubber/humidifier unit that permits air or other gases to be circulated through the scrubber/humidifier. The blower, scrubber, or humidifier can be part of the one or more of the multiple subunits within the building, or can be a unit that is brought into the one or more of the multiple subunits when decontamination of the subunits is desired.

In a particular example, the scrubber/humidifier unit includes an inlet and an outlet to establish a path of flow between the inlet and the outlet. The inlet transfers air present in the subunit of the building into the scrubber/humidifier unit, and the outlet transfers air or other gas from the scrubber/humidifier unit back into the subunit of the building, thereby circulating gas through the subunit. As described above, the blower can be part of the scrubber/humidifier unit to assist in the movement of gas through the scrubber/humidifier unit. In some examples, fans are placed in the subunit to increase circulation within the subunit.

One particular example of the disclosed building includes a central source of chlorine dioxide gas in fluid communication with one or more subunits in the building. The subunits include a humidifier, a blower, and a scrubber, wherein the blower, humidifier and the scrubber are a single unit. The unit includes a first inlet and a first outlet to establish a path of flow between first inlet and first outlet through the blower/scrubber/humidifier unit. The first inlet on the blower/scrubber/humidifier unit transfers air or other gas present in the subunit in the building into the blower/scrubber/humidifier unit where the air or other gas is exposed to the environment of the blower/scrubber/humidifier unit, and the first outlet transfers air or other gas from the blower/scrubber/humidifier unit out of the first outlet, thereby permitting circulation in at least one of the multiple subunits.

The foregoing and other features and advantages will become more apparent from the following detailed description of a several embodiments.

### BRIEF DESCRIPTION OF THE FIGURES

FIG. 1 is a schematic drawing showing an apparatus that can be used to decontaminate an article, such as laboratory equipment.

FIGS. 2 and 3 are digital images showing the front and back, respectively, of an embodiment of the apparatus of FIG. 1.

FIG. 4 is a schematic drawing showing an apparatus that can be used to decontaminate an area, such as a laboratory.

FIG. 5 is a schematic drawing showing a particular example of a scrubber/humidifier.

FIG. 6 is a schematic drawing showing a particular example of a system that can be used to transport chlorine dioxide gas from one room to another.

FIG. 7 is a digital image showing a mock bisafety cabinet.

FIG. 8 is a digital image showing a bisafety cabinet which was sealed with a piece of rubber.

### DETAILED DESCRIPTION

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. “Comprises” means “includes.” Hence “comprising A or B” means including A, or B, or A and B. Although methods and materials similar or equivalent to those described herein can be used in the practice or testing of the present disclosure, suitable methods and materials are described below. In addition, the materials, methods, and examples are illustrative only and not intended to be limiting.

Ambient: Condition of the environment, such as the temperature, humidity, or pressure, present within a sealed environment. In particular examples, ambient temperature in a sealed environment is about 62° F. to about 90° F. (such as about 65° F. to about 85° F., 68° F. to about 80° F., or 68° F. to about 72° F.), ambient humidity in a sealed environment is the humidity in the absence of supplemental humidification by a humidifier, and the ambient pressure in a sealed environment is the pressure in the absence of a vacuum.

Bacillus: A genus of bacteria whose collective features include degradation of most substrates derived from plant and animal sources, including cellulose, starch, pectin, proteins, agar, hydrocarbons, and others; antibiotic production; nitrification; denitrification; nitrogen fixation; facultative lithotrophy; autotrophy; acidophily; alkaliphily, psychrophily, thermophily and parasitism. Spore formation, universally found in the genus, is thought to be a strategy for survival in the soil environment, wherein the bacteria predominate. Aerial distribution of dormant spores likely explains the occurrence of Bacillus species in most habitats examined.

There are more than 40 recognized species in the genus Bacillus (Bergey's Manual of Systematic Bacteriology Vol 2 (1986)). These include, but are not limited to, B. acidocaldarius, B. alkalophilus, B. alvei, B. anthracis, B. azotoformans, B. badius, B. brevis, B. cereus, B. circulans, B. coagulans, B. fastidiosis, B. firmus, B. globisporus, B. insolitus, B. larvae, B. laterosporus, B. lentimorbus, B. lentus, B. licheniformis, B. macerans, B. macquariensis, B. marinus, B. megaterium, B. mycoides, B. pantothenticus, B. pasteurii, B. polymyxa, B. popillia, B. pumilus, B. schlegelii, B. sphaericus, B. stearothermophilus, B. subtilis, and B. thuringiensis. In one specific, non-limiting example, a Bacillus is Bacillus anthracis, the agent that causes anthrax.

Bacteria: Any of various prokaryotic organisms, including organisms within various phyla in the Kingdom Procaryotae. The terms encompass all microorganisms commonly regarded as bacteria, including Mycoplasma, Chlamydia, Actinomyces, Streptomyces, and Rickettsia. The term also includes cocci, bacilli, spirochetes, spheroplasts, protoplasts, and so forth.

Biological safety cabinet (BSC): A device that is designed to contain aerosols generated during work with infectious material through the use of laminar air flow and high efficiency particulate air (HEPA) filtration. Three types of BSCs (Class I, II, and III) are typically used in research laboratories. Open-fronted Class I and Class II BSCs are partial containment devices which provide a primary barrier offering significant levels of protection to laboratory personnel and to the environment when used in combination with good microbiological techniques.

Class I BSC provides protection to personnel and the environment from contaminants within the cabinet. It is suitable for work involving low to moderate risk pathogens needing containment, but not product protection.

Class II BSC protects the material(s) being manipulated inside the cabinet from external contamination, protects personnel and the environment. There are three basic types of Class II BSCs: Type A, Type B, and 100% exhaust. The major differences between the three types is in the percent of air exhausted or recirculated and the manner exhaust air is removed from the work area.

The gas-tight Class II BSC or glove box provides the highest attainable level of protection for personnel, environment, and product. It provides a total physical barrier between product and personnel. It is used when absolute containment of highly infectious or hazardous material is required.

Blower: A device that produces a current of air, and can be used to circulate air in an environment. A particular example includes a fan.

Central source: A facility that provides the primary (and in some examples only) location of one or more materials. For example, a central source of chlorine dioxide can be a room in a building where chlorine dioxide generators are kept, and can further include a source of dilute chorine, as well as conduits for transferring the chloride dioxide gas to other units in the building. A central source can be remote from the multiple units it supplies.

Chlorine dioxide (CO₂): A gas that is an extremely effective disinfectant, which rapidly inactivates pathogens such as bacteria, viruses, and parasites. Chlorine dioxide gas molecules can kill aerosolized, airborne pathogens, and also can diffuse through cracks and crevices in an article or a building or room and reach any surface that might have been reached by a pathogen. Chlorine dioxide gas has a greenish yellow color with a distinctive odor similar to that of chlorine. Chlorine dioxide is highly soluble in water but, unlike chlorine, chlorine dioxide does not react with water. It exists in aqueous solution as a dissolved gas.

A source of chlorine dioxide is any device that stores, releases, or produces chlorine dioxide. One type of a chlorine dioxide source is a chlorine dioxide generator. A chlorine dioxide generator is a device for producing chlorine dioxide gas, for example, a device that generates chlorine dioxide gas as needed. One such chlorine dioxide generator is the Saf-T-Chlor™ chlorine dioxide generator (CDG, Bethlehem, Pa.), which uses the reaction between dilute chlorine gas and thermally stable solid sodium chlorite to generate chlorine dioxide gas on demand. This reaction produces chlorine dioxide gas (in nitrogen), free of chlorite ion, chlorate ion or molecular chlorine.

Closed loop circulation system: A structure wherein the flow of materials between elements within the structure is restricted from the outside environment. For example, a close loop circulation system can include elements A, B and C, with fluid connections between A and B, B and C, and C and A, such that when materials (such as gases) flow through these connections from one element to the next, the materials do not contact the environment outside of the fluid connections or outside the elements.

Conduit: A passage through which materials, such as gases and liquids, can pass. Exemplary conduits include, but are not limited to, openings, pipes, tubes, or hoses. In particular examples, conduits can be used to transfer chlorine dioxide gas, for example from a source of chlorine dioxide to an environment contaminated or thought to be contaminated with a pathogenic spore. Similarly, conduits can be used to transfer humidified gas, such as humidified air, for example from a humidifier to an environment contaminated or thought to be contaminated with a pathogenic spore.

Decontamination: To substantially inactivate or remove unwanted pathogens or pathogenic spores, for example by killing substantially 100% of pathogens present.

Decontamination gas: A gas effective to kill or otherwise substantially eliminate the pathogenicity of a pathogen, such as a sporulated pathogen. One example includes, but is not limited to, chlorine dioxide gas.

Humidification: The process of increasing the relative humidity, for example by a humidifier. Examples of humidifiers include, but are not limited to evaporative humidifiers, steam humidifiers, and ultrasonic humidifiers. Humidity can be measured by a device known as a hygrometer.

Humidity: A measure of the amount of moisture present in a gas. Generally, the degree of humidity is expressed as relative humidity, or the ratio of the amount of water vapor in a gas at a specific temperature to the maximum amount that the gas could hold at that temperature, expressed as a percentage. A completely saturated gas is said to be at 100% relative humidity, and partial saturation is designated by smaller percentages, for example, 95%, 85%, 70%, 50%, or even less relative humidity.

Inhabitable structure: An object fit for occupation by humans. Examples of such structures include, but are not limited to, buildings (such as office or research buildings) and homes, as well as rooms thereof.

Negative pressure: Less than ambient atmospheric pressure.

Neutralize: To make chemically neutral. In a particular example, neutralizing chlorine dioxide gas involves reducing an amount of chlorine dioxide gas by exposing the gas to one or more agents that render the chlorine dioxide ineffective, such as a 10% NaOH/10% sodium thiosulfate hydrate solution in water.

Pathogen: Any of various bacteria, viruses, and protozoa can cause disease or death to humans, animals, or plants, or other biological organisms. Pathogenic spores are spores that are produced from a pathogen. Particular examples of pathogens that can produce spores include, but are not limited to, members of the genera Bacillus, Clostridium, Desulfotomaculans, Sporolactobacillus, and Sporpsarcina; members of the Phylum Apicomplexa (such as Plasmodium falciparum and Cryptosporidium parvum); and phytopathogenic fungi.

Portable: Capable of being easily or conveniently transported. In particular examples, a device is portable if it can be transported on a cart which is can be moved by 1 to 4 people, such as a cart with wheels. In some examples, a portable device is one weighing less than 500 pounds, such as less than 200 pounds, such as less than 100 pounds, for example a device weighing 50-450 pounds.

Rotometer: A device for measuring the rate of fluid flow. In some examples, a rotometer is a tapered, vertical tube having a circular cross section in which a float moves in a vertical path to a height dependent on the rate of fluid flow through the tube.

Seal: A substantially gas-impermeable closure. A sealed environment (such as a sealed article, sealed room, or sealed building) is one in which substantially all leaks have been blocked (for example, using plastic or other sheeting, tape, insulation, caulking, or combinations thereof) to form an environment that is substantially gas-impermeable. A sealed environment (such as a sealed article, sealed room, or sealed building) can include one or more ports that permit agents to be moved in and out of the sealed area.

Scrubber: A device that removes or neutralizes undesired agents. For example, scrubbers can be used to neutralize chlorine dioxide present in a sealed environment after decontamination. A particular example includes a chamber containing neutralizing agents, such as 10% NaOH/10% sodium thiosulfate hydrate.

Spore: A small, usually single-celled reproductive body that is highly resistant to desiccation and heat and is capable of growing into a new organism, produced especially by certain bacteria, fungi, algae, and non-flowering plants. Spores have proven to be the most durable type of cell found in nature, and in their cryptobiotic state of dormancy, they can remain viable for extremely long periods of time, perhaps millions of years. Spores do not form normally during active growth and cell division. Rather, their differentiation begins when a population of vegetative cells passes out of the exponential phase of growth, usually as a result of nutrient depletion. Typically, one spore is formed per vegetative cell. In some examples, the mature spore is liberated by lysis of the mother cell (sporangium) in which it was formed.

Mature spores have no detectable metabolism, a state that is described as cryptobiotic. They are highly resistant to environmental stresses such as high temperature (some endospores can be boiled for several hours and retain their viability), irradiation, strong acids, disinfectants, etc. Although cryptobiotic, they retain viability indefinitely such that under appropriate environmental conditions, they germinate into vegetative cells.

Viable: Capable of living, developing, or germinating under favorable conditions. For example, a viable pathogen is capable of developing under favorable conditions.

### Apparatus for Decontamination

Apparatuses are disclosed that can be used to decontaminate articles, such as laboratory equipment, as well as entire areas, such as a laboratory or building. In some examples, the apparatus is portable. For example, the apparatus can be on a cart (making the apparatus mobile) which can be moved by one to four people, such as one or two people. In some examples, the cart has dimensions of about 2 feet by 2 feet, about 2 feet by 4 feet, or about 4 feet by 4 feet. In particular examples, the apparatus is easily transported up or down a short flight of stairs (such as 3-4 steps). The apparatus is ideally compact enough to allow maneuverability in tight spaces, such as those found in laboratories.

In particular examples, the apparatus includes a source of chlorine dioxide gas and a humidifier on a cart, as well as an inlet conduit for introducing the chlorine dioxide gas and humidified air into the environment to be decontaminated, and an outlet conduit for removing air and chlorine dioxide gas from the environment. The apparatus can further include a blower in fluid communication with the humidifier to permit circulation of gas (such as air or chlorine dioxide) in the environment from the environment to the humidifier and from the humidifier to the environment. The apparatus can also include a scrubber for neutralizing the chlorine dioxide gas. In particular examples, the humidifier and the scrubber are a single unit.

The apparatus can include a first fluid flow path for transferring humidified air from the humidifier/scrubber to the sealed environment, a second fluid flow path for transferring chlorine dioxide gas from the source of the chlorine dioxide gas to the sealed environment, and a third fluid flow path for moving air from the sealed environment to the humidifier/scrubber via the blower. In some examples, the apparatus also includes a flow regulator, rotometer, or on/off valve in any of the fluid flow paths to regulate the flow of gas.

In certain examples, the apparatus also includes a dilute chlorine source and a fourth fluid flow path for transferring dilute chlorine gas to the chlorine dioxide generator. A flow regulator, rotometer, or on/off valve can be included in the fourth fluid flow path.

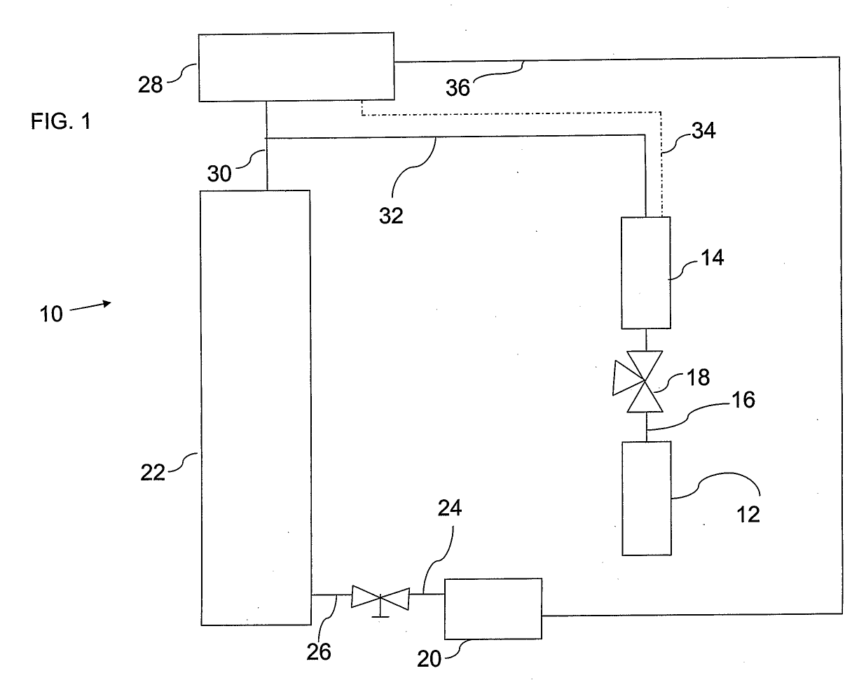

One particular example of the apparatus 10 is shown in FIG. 1. Apparatus 10 includes a diluted chlorine gas source 12 in fluid communication with a source of chlorine dioxide gas 14 and a first fluid flow path 16 for transferring diluted chlorine gas to a source of chlorine dioxide gas 14. First fluid flow path 16 includes a flow regulator 18, which regulates flow of the diluted chlorine gas to source of chlorine dioxide gas 14. First fluid flow path 16 can further include a pressure regulator or an on/off valve.

Apparatus 10 also includes a blower 20 in fluid communication with a humidifier/scrubber 22 and a second fluid flow path 24 for transferring air to humidifier/scrubber 22. Second fluid flow path 24 includes a flow regulator 26, which regulates the flow of air to scrubber 22. Second fluid flow path 24 can further include an on/off valve.

Humidifier/scrubber 22 and source of chlorine dioxide gas 14 include conduits to permit fluid communication with sealed environment 28. Sealed environment 28 is not part of the apparatus in this embodiment. Third fluid flow path 30 transports materials from humidifier/scrubber 22 to sealed environment 28, and fourth fluid flow path 32 or fifth fluid flow path 34 transports chlorine dioxide gas from source of chlorine dioxide gas 14 to sealed environment 28. In some examples, fluid flow path from source of chlorine dioxide gas 14 to sealed environment 28 is direct, as in fifth fluid flow path 34 (see dashed line in FIG. 1). In other examples, fourth fluid flow path 32 is in indirect connection with sealed environment 28 via third flow path 30 as shown in FIG. 1, thereby permitting mixing of chlorine dioxide gas with material from humidifier/scrubber 22. In some examples, fourth fluid flow path 32 or fifth fluid flow path 34 includes a rotometer or a flow regulator to regulate flow of the chloride dioxide gas to sealed environment 28. Sealed environment 28 is in fluid communication with blower 20. Sixth fluid flow path 36 transports air in sealed environment 28 to humidifier/scrubber 22 via blower 20 and second flow path 24. This creates a closed system whereby air is continually moving through sealed environment 28 and humidifier/scrubber 22. In some examples, third fluid path 30 includes a rotometer or a flow regulator to regulate flow of the humidified air to sealed environment 28.

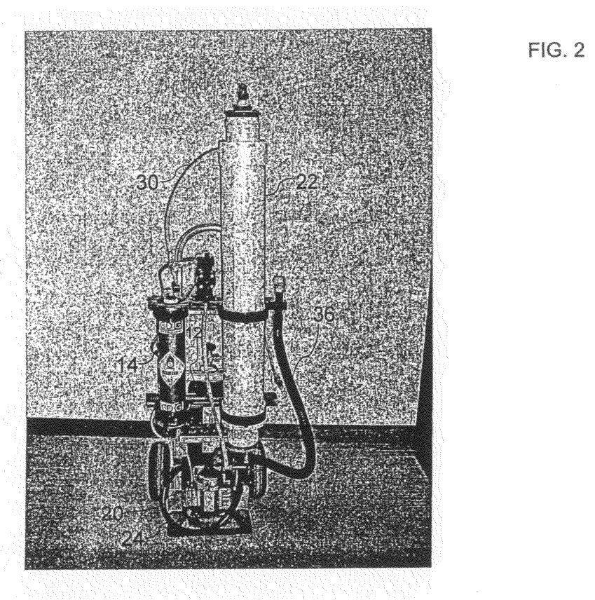

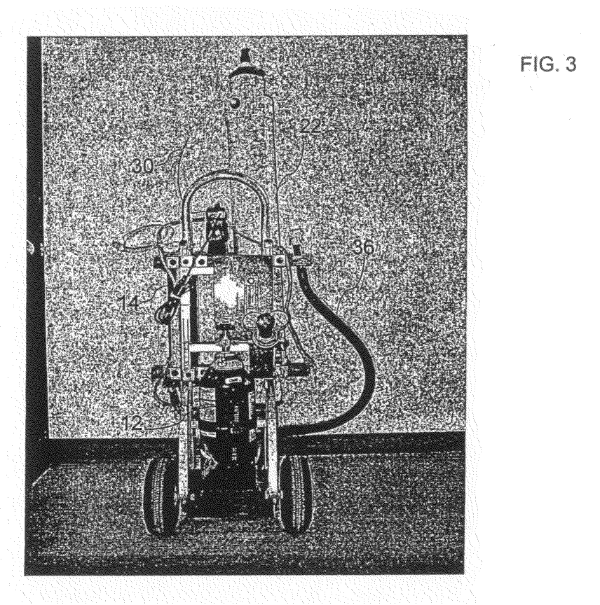

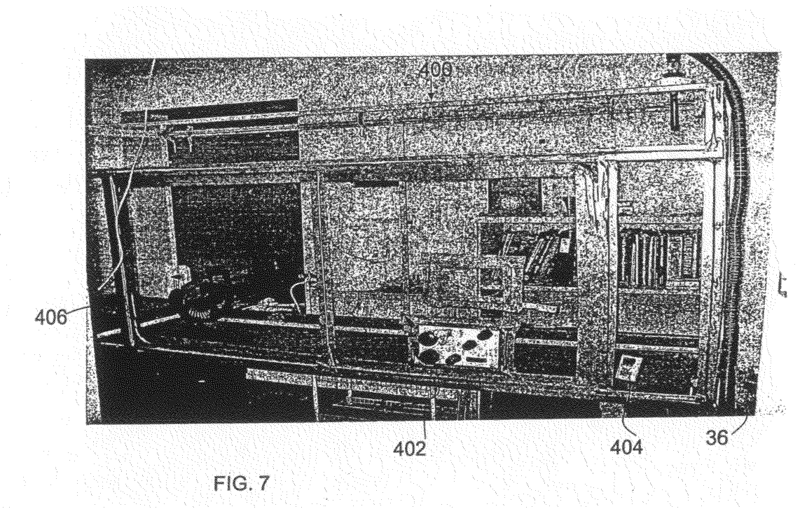

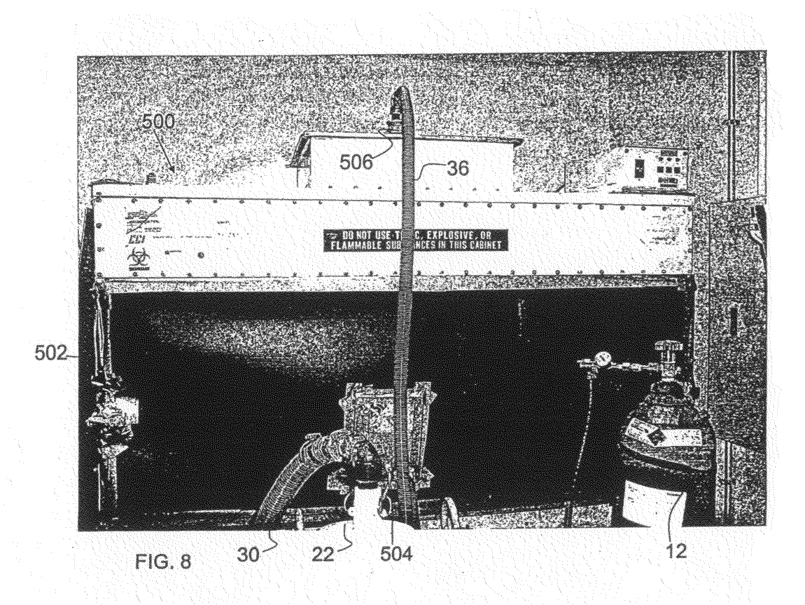

FIGS. 2 and 3 show the front and back, respectively, of a particular example of the apparatus shown in FIG. 1. The apparatus shown in FIGS. 2 and 3 is a portable, self-contained decontamination unit on a cart, which can be wheeled where needed and attached to an item to be decontaminated, such as a BSC or a room. For example, if the item to be decontaminated is a BSC, the cart could be wheeled to a sealed BSC (such as shown in FIGS. 7 and 8), and fluid flow path 36 attached to the sealed BSC to permit gas to be removed from the sealed BSC, and flow path 30 attached to the sealed BSC to permit introduction of gas (such as humidified air from humidifier/scrubber 22 and chlorine dioxide gas from chlorine dioxide source 14) into the sealed BSC. Water is added to humidifier/scrubber 22, and blower 20 turned on to initiate flow of humidified air from humidifier/scrubber 22 through fluid flow path 30, into the sealed BSC. A flow of dilute chorine gas is initiated from diluted chlorine gas source 12 to source of chlorine dioxide gas 14, thereby generating gaseous chlorine dioxide which is transported into sealed BSC via fluid flow path 32 and 30. Because of the closed loop nature of the system, gas is removed from the sealed BSC via fluid flow path 36, back into humidifier/scrubber 22. Once the sealed BSC has been exposed to gaseous chlorine dioxide for the predetermined time, flow of dilute chorine gas is terminated from diluted chlorine gas source 12, and a chlorine dioxide neutralization agent is added to humidifier/scrubber 22. Gas is allowed to continually circulate from sealed BSC to humidifier/scrubber 22 via fluid flow path 36 to permit neutralization of chlorine dioxide gas in sealed BSC, and back to sealed BSC from humidifier/scrubber 22 via fluid flow path 30, until the chlorine dioxide gas in sealed BSC is substantially neutralized.

In operation, an article in need of decontamination (such as a BSC) is sealed to form a gas-impermeable seal, thereby generating a sealed environment 28. Third fluid flow path 30 and fifth fluid flow path 34 or sixth fluid flow path 36 are attached to sealed environment 28, for example via one or more inlet conduits in sealed environment 28. Humidification of sealed environment 28 is initiated, for example if the ambient humidity of the sealed environment 28 is less than 70% humidity. Water is added to humidifier/scrubber 22, and air from blower 20 flows through second fluid flow path 24 to humidifier/scrubber 22, where the air is humidified. Humidified air flows from scrubber 22 to sealed environment 28 through third fluid flow path 30, and then out of sealed environment 28 through sixth fluid flow path 36 back to humidifier/scrubber 22 via second fluid flow path 24. The humidified air flows to permit sealed environment 28 to reach the desired relative humidity, which can be measured using a hygrometer. Humidification of sealed environment 28 enhances the susceptibility of pathogens to decontamination with chlorine dioxide gas. The desired relative humidity of the humidified air can be determined based on the particular characteristics of the article being decontaminated (such as the porosity of the article), the nature of the pathogen (such as the inherent ability of potential or actual pathogens to resist decontamination by chlorine dioxide), the concentration of chlorine dioxide gas to be used, and the relative humidity of the chlorine dioxide gas to be used.

During or after the humidification, a decontamination step begins. If no humidification is needed, the method can start at the decontamination step. Diluted chlorine gas 12 flows through first fluid path 16 to source of chlorine dioxide 14, where chlorine dioxide gas is generated. The resulting chlorine dioxide gas flows from source of chlorine dioxide 14 through fifth fluid flow path 34 directly to the sealed environment 28 or through fourth fluid flow path 32 and through third fluid flow path 30 where the chloride dioxide combines with the humidified air emitted from humidifier/scrubber 22 generating humidified chlorine dioxide gas, and the gas and then enters sealed environment 28 through third flow path 30. The particular concentration of chlorine dioxide in the carrier gas selected for use is a function of several factors, including, but not limited to, the ability of the particular pathogen to resist decontamination by chlorine dioxide, the duration of exposure to the chlorine dioxide gas, the humidity of sealed environment 28, the duration of the humidification step, and the relative humidity of the chlorine dioxide gas.

Sealed environment 28 is exposed to the chlorine dioxide gas for a predetermined period of time, which may be chosen based on a number of factors, including, but not limited to, the inherent ability of the particular pathogen to resist decontamination by chlorine dioxide, the concentration of chlorine dioxide gas, the humidity to which the article has been exposed during the humidification step, and the relative humidity of the chlorine dioxide gas. During decontamination, air continues to flow from humidifier/scrubber 22 through third flow path 30 to sealed environment 28, out of sealed environment 28 through sixth flow path 36 to blower, and then through second flow path 24 to humidifier/scrubber 22, resulting in air continually flowing though sealed environment 28.

After decontamination, a scrubbing step is initiated to neutralize the remaining chlorine dioxide gas in sealed environment 28. The flow of diluted chlorine gas 12 is terminated by flow regulator 18. A scrubbing solution is added to humidifier/scrubber 22, such as a 10% NaOH, 10% sodium thiosulfate hydrate solution in water. As the remaining chorine dioxide gas passes through sixth fluid flow path 36, and second fluid flow path 24, into humidifier/scrubber 22, the chlorine dioxide gas is neutralized by the scrubbing solution in humidifier/scrubber 22. Sealed environment 28 is scrubbed for a predetermined time, which is chosen based on a number of factors, including, but not limited to, the concentration of chlorine dioxide used in the decontamination step, the humidity of the sealed environment, and temperature of the sealed environment. The concentration of chlorine dioxide gas in sealed environment 28 can be monitored using a chlorine dioxide analyzer such as a spectrophotometer.

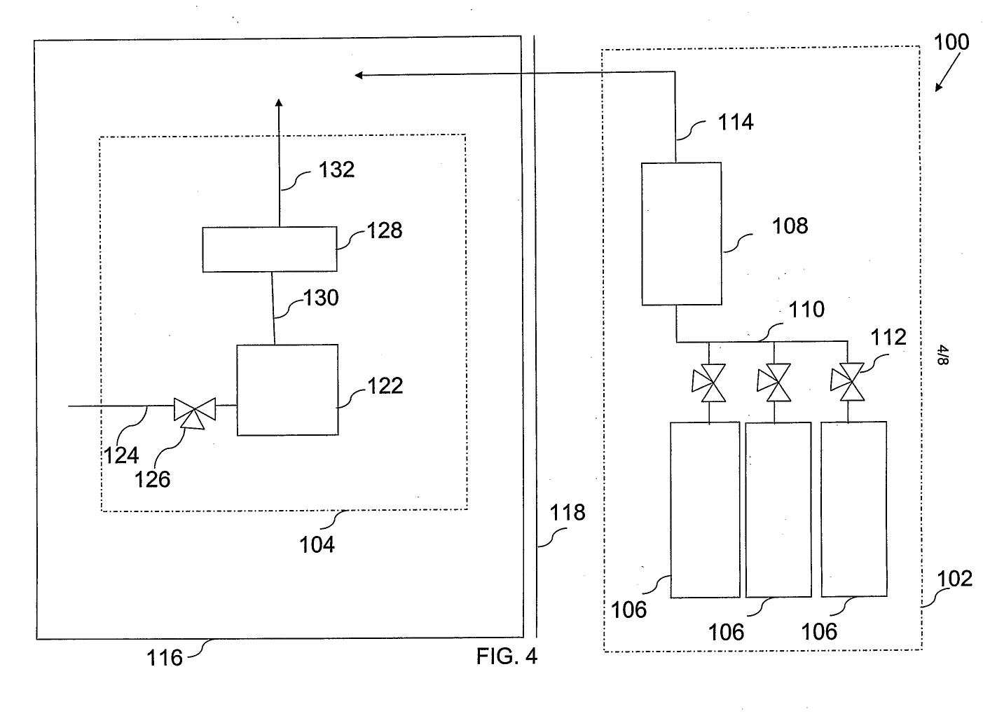

Another particular example of the apparatus 100 is shown in FIG. 4. This example extends the principles of apparatus 10 to decontaminating entire areas, such as an inhabitable structure, such as a building (for example a laboratory). Apparatus 100 includes at least two units, a chloride dioxide source unit 102, and a scrubber/air flow unit 104. Each unit can be portable, for example on a cart which can be stored until use. In some examples, each unit 102, 104 is on a separate cart, and in other examples the units are on a single cart. When needed, scrubber/air flow unit 104 can be brought into an area to be decontaminated. Alternatively, scrubber/air flow unit 104 can be part of the area, such as a laboratory. Similarly, chloride dioxide source unit 102 can be brought to a room having a fluid flow path into the area to be decontaminated. Alternatively, chloride dioxide source unit 102 is a remote unit that is stored in a room having one or more fluid flow paths into one or more areas to be decontaminated.

In particular examples, source of chlorine dioxide gas 108 is a chloride dioxide generation unit, and chloride dioxide source unit 102 includes one or more diluted chlorine sources 106 in fluid communication with source of chlorine dioxide gas 108 and first fluid flow path 110 for transferring diluted chlorine gas to source of chlorine dioxide gas 108. First fluid flow path 110 includes flow regulator 112, which regulates flow of diluted chlorine gas to source of chlorine dioxide gas 108. Source of chloride dioxide gas 108 also includes a second fluid flow path 114, which transports chlorine dioxide gas into sealed environment 116 containing scrubber/air flow unit 104, for example through wall 118. First fluid flow path 110 can further include a pressure regulator and an on/off valve.

Scrubber/air flow unit 104 includes a third fluid flow path 124 which transports air present in sealed environment 116 into humidifier/scrubber 122. Third fluid flow path 124 can optionally include a flow regulator 126, an on/off valve, or a rotometer, for example to regulate flow of air to humidifier/scrubber 122.

Humidifier/scrubber 122 is in fluid communication with a blower 128 and a fourth fluid flow path 130 for transferring air from humidifier/scrubber 122 to blower 128. Blower 128 is fluid communication with sealed environment 116 and a fifth fluid flow path 132, which transports air out of humidifier/scrubber 122 into sealed environment 116. Fourth or fifth fluid flow path 130 can optionally include an on/off valve, rotometer, or flow regulator to regulate flow of air to sealed environment 116. Sealed environment 116 can also include a hygrometer for determining relative humidity of sealed environment 116, and a chloride dioxide sensor to determine the concentration of chlorine dioxide in sealed environment 116. Sealed environment 116 can further include fans or blowers to promote circulation in sealed environment 116.

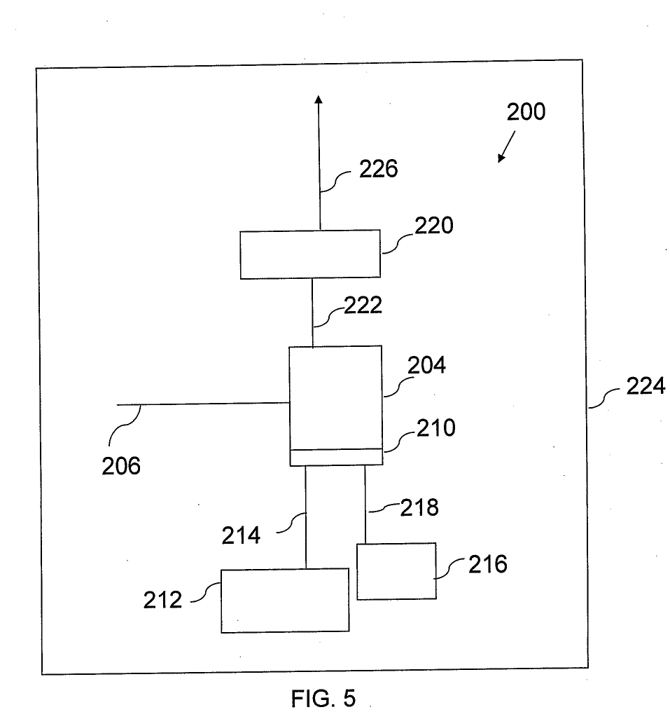

A particular example of a scrubber/air flow unit is shown in FIG. 5. Scrubber/air flow unit 200 includes humidifier/scrubber 204 in fluid communication with sealed environment 224, and a first fluid flow path 206 which transports air from sealed environment 224 into humidifier/scrubber 204. First fluid flow path 206 can optionally include an on/off valve, for example to regulate flow of the humidified air to humidifier/scrubber 204.

Humidifier/scrubber 204 includes compartment 210, which can be used to hold agents, such as water or chloride dioxide neutralization agents. Compartment 210 is in fluid communication with neutralization reservoir 212 and a fourth fluid flow path 214 which transports chloride dioxide neutralization agents into compartment 210 of humidifier/scrubber 204. In particular examples, neutralization reservoir 212 has at least a 10 gallon capacity. Compartment 210 is also in fluid communication with a water reservoir 216 and a fifth fluid flow path 218 which transports water into compartment 210 of humidifier/scrubber 204 for humidification of air entering humidifier/scrubber 204 from first fluid flow path 206. In particular examples, water reservoir 216 has at least a 3 gallon capacity. In some examples, fifth fluid flow path 218 enters fourth fluid flow path 214 prior to entering compartment 210. A pump can be used to move materials in and out of compartment 210 from neutralization reservoir 212 and water reservoir 216. Fourth fluid flow path 214 or fifth fluid flow path 218 can optionally include an on/off valve, rotometer, or flow regulator to regulate flow of the neutralizer or water to compartment 210.

Humidifier/scrubber 204 is also in fluid communication with blower 220 and sixth fluid flow path 222 for transferring air to blower 220. Air is transported from humidifier/scrubber 204, to blower 220, and exits blower via seventh fluid flow path 226 into sealed environment 224. Sixth 222 or seventh 226 fluid flow path can optionally include an on/off valve, rotometer, or flow regulator to regulate flow of air to sealed environment 224.

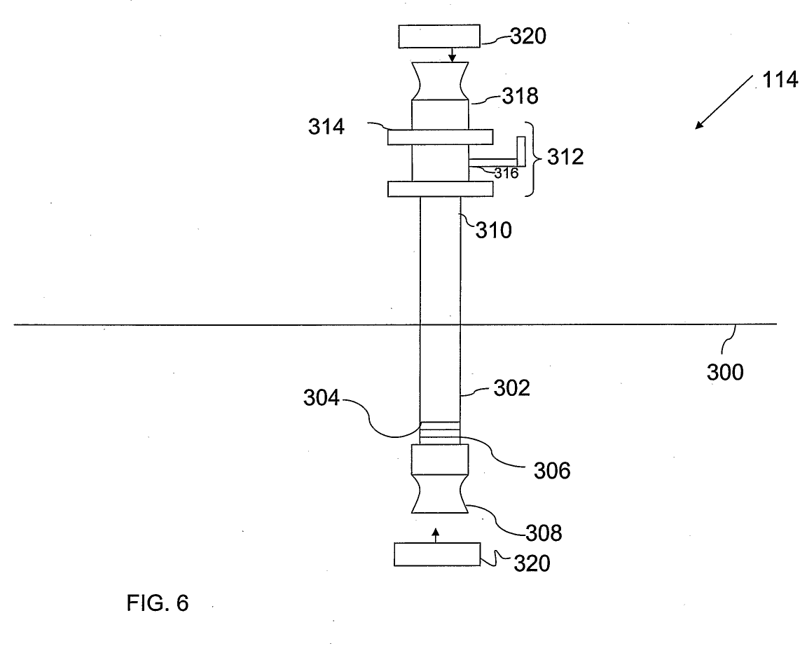

A particular example of second fluid flow path 114 of chloride dioxide generation unit 102 (FIG. 4) is shown in FIG. 6. Second fluid flow path 114 transports chlorine dioxide from one area to another, such as from one room to another, for example through wall 300. Second fluid flow path 114 includes pipe (or other conduit) 302 having threads 304 at one end 306. End 306 includes a female adapter with quick disconnect fitting 308. In particular examples, female adapter with quick disconnect fitting 308 is a polypropylene 1 inch female adapter with quick disconnect fitting (such as Ryan Herco part number 1302-010). Opposite end 310 includes ball valve 312 having one side threaded 314 and one side socket 316. A particular example of ball valve 312 is a 1 inch PVC ball valve, (such as Ryan Herco part number 4904-010). Socket side 316 of ball valve 312 can be solvent welded to top of pipe 302. Threaded side 314 of ball valve 312 is fitted with a polypropylene quick disconnect male adapter 318, such as Ryan Herco part number 1301-010. Quick disconnect female adapter 308 and quick disconnect male adapter 318 can be capped with cap 320 when materials are not being transported through pipe 302. Exemplary caps include, but are not limited to a polypropylene quick disconnect dust cap (such as Ryan Herco part number 1314-010). In particular examples, pipe 302 is 1 inch PVC pipe. In some examples, pipe 302 protrudes at least 1 inch from both sides of wall 300.

In operation, an area in need of decontamination, such as a room or building is sealed to form a substantially gas-impermeable seal, thereby generating a sealed environment 116 (FIG. 4). For example, if a room is to be decontaminated, doors and windows are closed, and cracks leading to the outside of the room are sealed, for example with tape or caulking. Referring to FIG. 4, humidification of sealed environment 116 is initiated, for example if the ambient humidity of the sealed environment 116 is less than about 70%. In particular examples, if the ambient humidity of the sealed environment 116 is at least 70%, the humidification step is omitted. If increased humidification of the sealed environment 116 is desired, water is introduced to humidifier/scrubber 122, and air from sealed environment 116 or from an air source flows through third fluid flow path 124 to humidifier/scrubber 122, where air is humidified. Humidified air flows from humidifier/scrubber 122 to blower 128 through fourth fluid flow path 130, and then out of blower 128 into sealed environment 116 through fifth fluid flow path 132. In some examples, humidifier/scrubber 122 and blower 128 are connected such that humidified air flows directly from humidifier/scrubber 122 to blower 128 (or blower 128 is part of humidifier/scrubber 122), and then out of blower 128 into sealed environment 116 through fifth fluid flow path 132. The humidified air flows to permit sealed environment 116 to reach the desired relative humidity, which can be measured using a hygrometer. The relative humidity of the humidified air and the duration of incubation can be determined based on the particular characteristics of the area being decontaminated, including, but not limited to, the size of the area, the inherent ability of potential or actual pathogens to resist decontamination by chlorine dioxide, the concentration of chlorine dioxide gas to be used, and the relative humidity of the chlorine dioxide gas to be used.

During or after humidification, a decontamination step begins. However, if no supplemental humidification was provided, the method can start with the decontamination step. Second fluid flow path 114 is attached to the sealed environment 116 or to the source of chlorine dioxide 108 as needed. Diluted chlorine gas 106 is permitted to flow through first fluid path 110 to source of chlorine dioxide gas 108, where the chlorine dioxide is generated. The resulting chlorine dioxide flows from source of chlorine dioxide 108 to through second fluid flow path 114 to the sealed environment 116. For example, source of chlorine dioxide gas 108 can be connected to sealed environment 116 using the attachment shown in FIG. 6. Referring to FIGS. 4 and 6, cap 320 is removed, and source of chlorine dioxide gas 108 attached to quick disconnect fitting 308. This permits gaseous chlorine dioxide to flow through fluid flow path 302 into the area in need of decontamination 116.

The particular concentration of chlorine dioxide in the dilute chlorine gas selected for use is a function of several factors, including, but not limited to, the ability of the particular pathogen to resist decontamination by chlorine dioxide, the duration of exposure to the chlorine dioxide gas, the humidity of sealed environment 116, and the duration of the humidification step.

Sealed environment 116 is exposed to the chlorine dioxide gas for a predetermined period of time, which is chosen based on a number of factors, including, but not limited to, the inherent ability of the particular pathogen to resist decontamination by chlorine dioxide, the concentration of chlorine dioxide gas, the humidity to which the area has been exposed during the humidification step, and the relative humidity of the chlorine dioxide gas. During the decontamination step, air in sealed environment 116 is continually circulated through humidifier scrubber 122 via third fluid flow path 124, fourth fluid flow path 130, and fifth fluid flow path 132.

After the decontamination step, a scrubbing step is initiated to neutralize the remaining chlorine dioxide gas in sealed environment 116. The flow of diluted chlorine gas 106 is terminated by flow regulator 112 (or by a regulator in second fluid flow path 114). A neutralizing solution is introduced to humidifier/scrubber 122, such as a 10% NaOH, 10% sodium thiosulfate hydrate solution in water. As the remaining chorine dioxide gas passes through third fluid flow path 124 into humidifier/scrubber 122, the chlorine dioxide gas is neutralized by the neutralizing solution in humidifier/scrubber 122. Neutralized air is introduced into sealed environment via fifth fluid flow path 132. Sealed environment 116 is scrubbed for a predetermined time, which is chosen based on a number of factors, including, but not limited to, the concentration of chlorine dioxide used in the decontamination step and the humidity of the sealed environment. The concentration of chlorine dioxide gas in the sealed environment 116 can be monitored using a chlorine dioxide analyzer such as a spectrophotometer.

### Methods of Decontamination

Decontamination methods are disclosed for decontaminating pathogens. The methods can be used to decontaminate an article (such as laboratory equipment, for example incubators, centrifuges, biological safety cabinets, refrigerators, freezers, water baths, work benches, glassware, and so on) or area (such as a building and sub-sections thereof, such as rooms and hallways, as well as cargo holds on transportation vessels such as airplanes, trucks, trains and ships.) that is actually or potentially contaminated with one or more pathogens. In some examples, the method is capable of decontaminating an area that is at least 20 ft×40 ft×9 ft.

Unlike many conventional methods of decontamination, which can take many hours or days to complete, require significant amounts of laboratory down time, and use burdensome equipment, the present method can use a portable decontamination apparatus which is used to decontaminate an article or area, from start to finish, in just a few hours.

In particular examples, the method includes providing a sealed environment, humidifying the sealed environment and introducing chlorine dioxide gas to the sealed environment to kill substantially 100% of pathogens present. During the humidification and introduction of chlorine dioxide gas, the humidified air and chlorine dioxide gas are continuously circulated through the sealed environment using a closed loop circulation system. In some examples, the method does not include humidifying the sealed environment. For example, if the ambient humidity of the sealed environment is at least 70%, the humidification step can be omitted.

If the environment to be decontaminated is an article, such as a BSC, the article can be sealed by closing or sealing any openings in the article, to thereby convert the interior of the article into a substantially sealed environment. Alternatively, the article itself can be enclosed in a substantially sealed environment. However, conduits can be made (or left in) to permit influx and efflux of humidified air, chlorine dioxide gas, or other agents to and from the sealed environment. The sealed environment is humidified if necessary, for example to achieve a relative humidity of at least 70% but no more than 90%, such as a relative humidity of 70-90%, such as 80-85%. During or after the humidification, a decontamination step is performed by introduction of chlorine dioxide gas into the sealed environment. However, if there was no humidification step, the sealed environment can proceed with the decontamination step. The sealed environment is exposed to the chlorine dioxide gas for a time and at a concentration sufficient to kill substantially 100% of the pathogens on the article or in the area. In particular examples, the method further includes removing excess chlorine dioxide gas after the decontamination step by exposing the gas to a neutralizing composition for a time sufficient to significantly decrease the amount of chlorine dioxide present in the sealed environment.

Examples of such articles that can be decontaminated with the disclosed method include, but are not limited to, laboratory equipment, such as BSCs, refrigerators, centrifuges, incubators, water baths, and so on. In some examples, the method is used to decontaminate an entire area, such as a laboratory, or an entire building.

In certain other examples, the area is a room (such as a laboratory). After the room is sealed to generate a sealed environment (for example by closing doors and windows and sealing any cracks), the room is humidified to at least 70% relative humidity, exposed to at least 1000 ppm chlorine dioxide for one hour, and then the chlorine dioxide gas is neutralized. In particular examples, the area is a building, and the method includes sealing the building, humidifying the sealed building to at least 70% relative humidity, exposing the sealed building to at least 1000 ppm chlorine dioxide for one hour, and then neutralizing the chlorine dioxide gas. In yet other examples, the method is a method of decontaminating an article, and the method includes sealing the article, humidifying the sealed article to at least 70% relative humidity, exposing the sealed article to at least 1000 ppm chlorine dioxide for one hour, and then neutralizing the chlorine dioxide gas. Such methods can be used to decontaminate the area or article by killing substantially 100% of pathogens present in the area or on the article. It is noted that if the ambient humidity of the sealed environment is already at 70% relative humidity, additional humidification can be omitted if desired. In particular examples, humidification, decontamination, and neutralization take less than two hours to complete.

### Sealed Environment

A substantially sealed environment is provided prior to beginning the disclosed decontamination method. To generate a sealed environment, openings in the article or area to the outside environment are sealed. For example, if the article to be decontaminated is a BSC, the openings in the BSC are sealed, for example using plastic sheeting and tape. Similarly, if the article to be decontaminated is a refrigerator, freezer or incubator, the opening to the refrigerator, freezer, or incubator can be sealed, for example by using plastic sheeting and substantially gas impermeable tape. Alternatively, if the article is small, such as a table-top centrifuge or water bath, the article can be placed into a decontamination chamber capable of being sealed. A decontamination chamber generally is a substantially gas-impermeable environment. Examples of decontamination chambers include, but are not limited to, sealed, rigid containers, and rooms or buildings that have been sealed to substantially prevent the influx or efflux of gas.

In particular examples, an area is to be decontaminated. Specific examples of areas which can be decontaminated using the disclosed methods include, but are not limited to, individual rooms or areas within a building, such as a laboratory, as well as an entire building. In such examples, the area to be decontaminated is sealed to prevent the escape of chlorine dioxide to the atmosphere. Examples of methods that can be used to seal an area include, but are not limited to, closing doors and windows, sealing cracks that lead out of the area to be decontaminated (for example with expanding foam or silicone caulking), and sealing openings with foil-backed foam insulation or with poly-sheeting and tape.

Conduits can be introduced into the sealed environment to permit influx and efflux of desired agents to and from the sealed environment. For example, tubing or piping can be introduced via a port in the sealed environment. Ideally, such tubing or piping does not adversely interact with the materials that flow through it, such as chlorine dioxide gas.

### Humidification

A sealed environment is humidified to enhance the activity of the chlorine dioxide gas. If the desired level of relative humidity is present (such as an ambient humidity of 70-90%), no additional humidification is needed. However, if the desired level of relative humidity is not present in the sealed environment, the relative humidity can be increased. The relative humidity chosen and the duration of exposure to the relative humidity can be optimized to suit a particular decontamination project, and can vary depending on, for example, the type of decontamination area or article to be decontaminated, the concentration of chlorine dioxide gas used, and/or the length of time the article is exposed to the chlorine dioxide gas. Generally, the degree of humidity is expressed as relative humidity, or the ratio of the amount of water vapor in a gas at a specific temperature to the maximum amount that the gas could hold at that temperature, expressed as a percentage. A completely saturated gas is said to be at 100% relative humidity, and partial saturation is designated by smaller percentages, for example, 95%, 85%, 75%, 50%, or even less relative humidity.

In particular examples, the desired amount of relative humidity in the sealed environment is at least 70%, and in some examples no more than 90%. Particular amounts include, but are not limited to, at least 75%, at least 80%, or at least 85%. In some examples, the sealed environment is humidified to 70-90%, 75-85%, or 80-89% relative humidity. In particular examples, the desired level of humidity is maintained throughout the decontamination process. Therefore, in certain examples, the sealed environment is exposed to at least 70% humidity for at least 1 hour, or at least 2 hours. In some examples, the humidification step is carried out at ambient temperature (such as about 68° F.-72° F.), although lower or higher temperatures can be employed if desired. The amount of relative humidity can be determined and regulated using a commercially available humidity gauge (hygrometer).

Humidification of the sealed environment can be achieved as follows. Water is introduced into a chamber (such as a humidifier), and then air is passed through the chamber (for example with a fan or blower), thereby generating humidified air. The amount of water added to the chamber will depend on the starting relative humidity of the sealed environment; the greater the starting relative humidity, the less water added to the chamber. The humidified air is introduced into the sealed environment, for example via a tube, hose, or pipe, thereby humidifying the sealed environment. Although the humidification step generally is carried out using humidified air, other humid gases, such as humidified nitrogen gas, can be used.

In a particular example, water is introduced into a chamber, such as a humidifier/scrubber. A blower is used to move air past the water, thereby generating humidified air. The resulting humidified air is forced by the blower into the sealed environment, for example via a tube or pipe. The air circulation loop is closed by passing air from the sealed environment back into the humidifier/scrubber. Therefore, air is continuously cycled between the scrubber and the sealed environment. In a particular example the humidifier/scrubber is a 6 inch×36 inch or an 18 inch×36 inch column packed with column packing having a high surface area to volume ratio for mass transfer from liquid to gas (for example FLEXIPAC® or FLEXERAMIC® from Koch-Glitsch, Wichita, Kans.). A hygrometer can be placed in the sealed environment to measure relative humidity.

### Decontamination

During humidification or after the sealed environment is at the desired level of relative humidity, such as 70-90% relative humidity, chlorine dioxide gas is introduced into the sealed environment. Under certain reaction conditions, chlorine dioxide inactivates pathogens. Chlorine dioxide gas is an excellent decontaminating agent because the gas molecules can kill aerosolized, airborne pathogens, and can diffuse through cracks and crevices in an article or a room or building and reach any surface that might have been reached by the target pathogen.

Because chlorine dioxide gas is unstable at high concentrations, it is usually generated at the point of use. The chlorine dioxide gas can be prepared by any method known in the art. One such method involves passing a stream of diluted chlorine gas (examples include, but are not limited to, air-diluted chlorine gas and nitrogen-diluted chlorine gas) at a metered rate through a column of finely divided sodium chlorite, and into a partially evacuated chamber. This procedure is described more fully in Grubitsch et al. (Monatsh., 93:246, 1962).

Another method of preparing chlorine dioxide gas is the reaction of sodium chlorite solutions in the presence of acids. In one example, a dilute solution of aqueous potassium persulfate is treated with a dilute solution of aqueous sodium chlorite at ambient temperatures (20-30° C.) in a closed reaction vessel. This method is discussed more fully in Rosenblatt et al. (J. Org. Chem., 28:2790, 1963).

In some examples, the chlorine dioxide gas is delivered via a chlorine dioxide source, which can be any device that stores, releases, or produces chlorine dioxide. One type of a chlorine dioxide source is a chlorine dioxide generator. A chlorine dioxide generator is a device for producing chlorine dioxide gas, for example, a device that generates chlorine dioxide gas as needed. One such chlorine dioxide generator is the CDG Gas:Solid™ chlorine dioxide generator (CDG, Bethlehem, Pa.), which uses the reaction between dilute chlorine gas (such as a nitrogen-diluted chlorine gas) and thermally stable solid sodium chlorite (Saf-T-Chlor™) to generate chlorine dioxide gas on demand. CDG Gas:Solid chlorine dioxide generators are available in at least two sizes: bench-scale generators for smaller scale applications (such as decontamination of laboratory equipment), and plant-scale generators which are useful for providing chlorine dioxide in amounts sufficient to decontaminate large areas, such as a laboratory or a building.

In some examples, the chlorine dioxide gas is delivered to the sealed environment in the form of a gaseous mixture of chlorine dioxide and an inert carrier gas, such as nitrogen gas or air.

The concentration of chlorine dioxide can be varied to suit the needs of a particular decontamination project. The amount of chlorine dioxide used will kill substantially 100% of the pathogens present on the article or in the area. For example, in some examples the concentration of gaseous chlorine dioxide is at least 1000 ppm, such as at least 2000 ppm, at least 2500 ppm, at least 3000 ppm, at least 3500 ppm, or even at least 4000 ppm, for example 1000-4000 ppm or 2500-4000 ppm gaseous chlorine dioxide. In some examples, the chlorine dioxide exposure time is adjusted. For instance, in some examples, the article or area is exposed to the gaseous chlorine dioxide for at least 15 minutes, at least 30 minutes, or at least 60 minutes. In some examples, the article or area is exposed to the gaseous chlorine dioxide for no more than 60 minutes, such as 15-60 minutes. The particular concentration of chlorine dioxide in the carrier gas selected for use is a function of several factors, including the inherent ability of the particular pathogen to resist decontamination by chlorine dioxide, the duration of exposure to the chlorine dioxide gas, the relative humidity of the sealed environment, the duration of the humidification step, and the relative humidity of the chlorine dioxide/carrier gas.

In particular examples, decontamination of the sealed environment is achieved as follows. A dilute chlorine gas (such as a gas containing 4% chlorine 96% nitrogen) is introduced into a cartridge containing granular solid sodium chlorite, such as the CDG Saf-T-Chlor™ thermally stable sodium chlorite (CDG, Bethlehem, Pa.). The emitted chlorine dioxide is introduced into the humidified sealed environment, for example via conduit. In some examples, the chlorine dioxide gas is introduced directly into the sealed environment. In other examples, chlorine dioxide is first exposed to humidified air, and then introduced into the sealed environment. Once in the sealed environment, the chlorine dioxide gas can decontaminate pathogens present in the sealed environment. The air in the sealed environment, including the chlorine dioxide gas, is circulated through a chamber as described above in the humidification step. In particular examples, at least 1000 ppm chlorine dioxide gas is circulated through the sealed environment for 15-60 minutes.

### Neutralization of Chlorine Dioxide

Following the decontamination step, chlorine dioxide gas in the sealed environment is neutralized. Flow of the dilute chlorine gas is terminated, thus stopping production of the chlorine dioxide gas. The chamber previously used for humidification can be used to “scrub” the chlorine dioxide gas contaminated sealed environment clean as follows. A neutralization agent is added to the chamber. Any agent that neutralizes chlorine dioxide gas can be used. In one example, the agent is a solution of 10% NaOH, 10% sodium thiosulfate hydrate, in water. As described above, air continually circulates between the sealed environment and the chamber. After introduction of the neutralization agent into the chamber, air coming from the sealed environment that contains chlorine dioxide gas will pass through the neutralization agent, thus neutralizing the chlorine dioxide gas. The air emitted into the sealed environment from the chamber will have a reduced concentration of chlorine dioxide gas.

Chlorine dioxide-contaminated air is passed through the chamber, until the level of chlorine dioxide gas is significantly reduced, for example to a level of less than 1 ppm chlorine dioxide gas, such as less than 0.5 ppm chlorine dioxide gas, or less than 0.1 ppm chlorine dioxide gas. Methods for measuring chlorine dioxide gas concentration are known.

For example, devices are commercially available that measure levels of chlorine dioxide (for example from Optek Danlut (Germantown, Wis.), Scott Instruments (Exton, Pa.) and PureAire Monitoring Systems, Inc. (Rolling Meadows, Ill.)). Such devices can be placed in the sealed environment to measure the chlorine dioxide gas concentration in the sealed environment. Another method that can be used to monitor the reduction in chlorine dioxide gas concentration is to monitor the colorimetric reaction that occurs between the chlorine dioxide gas and a neutralizing agent.

### EXAMPLE 1

### Decontamination of a Mock Biological Safety Cabinet

This example describes methods used to decontaminate a mock BSC containing test strips with pathogenic spores, using the apparatus shown in FIGS. 2 and 3. One skilled in the art will appreciate that similar methods can be used to decontaminate other pieces of laboratory equipment, or entire rooms or buildings.

Test strips contaminated with 2.4×10⁴ B. subtilis var. niger spores (Castle Biospore Biological Indicators, Castle, Inc., Rochester, N.Y.) were placed inside a mock BSC (FIG. 7) in various positions. The mock BSC was generated such that it was sealed from the outside environment, and included two openings to permit influx and efflux of air and other agents through the mock BSC.

After sealing the mock BSC, the sealed mock BSC was humidified to 74-95% relative humidity using the following methods. The cart shown in FIGS. 2 and 3 was wheeled up to the mock BSC shown in FIG. 7. Referring to FIGS. 2, 3 and 7, water was added to a humidifier/scrubber 22, and a blower 24 attached to the humidifier/scrubber 22 to allow air to pass by the water to generate humidified air. A hose 30 coming from the humidifier/scrubber which emitted humidified air was introduced into one of the openings 402 in the mock BSC 400. A hose 36 coming out of the mock BSC was attached to the blower 22, thereby providing a closed-loop which circulated humidified air from the humidifier/scrubber 22 into the mock BSC 400, out of the mock BSC 400 and back to the humidifier/scrubber 22, and so on. A hygrometer 404 in the mock BSC was used to monitor the relative humidity in the mock BSC.

During the humidification, chlorine dioxide gas was introduced into the mock BSC as follows. A tank 12 containing a 4% chlorine/96% nitrogen gas mixture was turned on to permit the flow of gas to a CDG Saf-T-Chlor™ chlorine dioxide gas generator 14. This generator includes a sodium chlorite cartridge in fluid communication with the 4% Cl/96% N₂ gas mixture. The resulting chlorine dioxide gas was allowed to flow into the hose 30 exiting the humidifier/scrubber (containing the humidified air), and was allowed to enter the mock BSC along with the humidified air. The mock BSC was exposed to 1500-2200 ppm chlorine dioxide gas for 60-90 minutes as shown in Table 1. A chlorine dioxide monitor in the mock BSC allowed for a determination of the amount of chloride dioxide in the mock BSC. In some instances, the air circulation was increased by including a fan 406 in the mock BSC.

Following exposure to chlorine dioxide, the tank 12 containing a 4% Cl/96% N₂ gas mixture was turned off, to stop the flow and production of chlorine dioxide gas. To neutralize the chlorine dioxide gas, the following methods were used. A neutralizing solution that included 10% NaOH and 10% sodium thiosulfate hydrate (in water) was introduced into the humidifier/scrubber 22. Air from the mock BSC containing chlorine dioxide gas was circulated through the closed system described above, such that when the air containing chloride dioxide gas passed through the humidifier/scrubber 22, it was at least partially neutralized. The air was allowed to circulate from the mock BSC, into the humidifier/scrubber, into the mock BSC, and back to the humidifier/scrubber (and so on) until the concentration of chloride dioxide reached less than 0.1 ppm chloride dioxide. The chlorine dioxide monitor in the mock BSC allowed for a determination of the amount of chloride dioxide in the mock BSC.

The test strips were cultured under permissive culture conditions (15, 24, 28 and 72 hour incubation in tryptic soy broth) to determine whether the spores were viable following the decontamination protocol. As shown in Table 1, all spores on the test strip were killed using the disclosed apparatus and method.

TABLE 1
Decontamination of a mock BSC.

| | Ramp | | Exposure | Scrub | Relative | | Total | |
|---|---|---|---|---|---|---|---|---|
| | Up | [ClO₂] | Time | time | humidity | Temp | Time | |
| Run | (min)* | (ppm) | (min)$ | (min)\hat{ } | (%) | (° F.) | (min) | Kill# |
| A | 10 | 1500 | 90 | 30 | 95 | 69 | 130 | 16/16 |
| B | 6 | 1500 | 90 | 33 | 85 | 69 | 129 | 16/16 |
| C | 5 | 2200 | 60 | 3 | 74 | 69 | 68 | 17/17 |
| D | 6 | 2000 | 60 | 3 | 75 | 68 | 69 | 17/17 |

*amount of time it took to achieve desired humidity and ClO₂ concentration.
$amount of time mock BSC exposed to the noted ClO₂ concentration.
\hat{ }amount of time it took to neutralize gas to below 0.1 ppm.
#number of test strips negative for spore growth.

### EXAMPLE 2

### Decontamination of a Biological Safety Cabinet (BSC)

This example describes methods used to decontaminate a BSC containing test strips with pathogenic spores, using the apparatus shown in FIGS. 2 and 3. One skilled in the art will appreciate that similar methods can be used to decontaminate other pieces of laboratory equipment, or entire rooms or buildings.

Test strips described in Example 1 were placed inside a BSC (FIG. 8) in various positions. The cart shown in FIGS. 2 and 3 was wheeled up to the BSC shown in FIG. 8. Referring to FIGS. 2, 3 and 8, the opening in the front of the BSC 500 was sealed using a polyethylene sheet 502 or plastic sheeting and duct tape. An opening 504 was made in the plastic or polyethylene 502 to permit influx of air and other agents through the sealed BSC 500. Gas was evacuated from the sealed BSC using the port 506 on the top of the sealed BSC through fluid flow path 36.

After sealing the BSC, the sealed area was humidified to 75% relative humidity as described in Example 1. Briefly, water was added to a humidifier/scrubber 22, and a blower 24 attached to the humidifier/scrubber 22 to allow air to pass by the water to generate humidified air. A hose 30 coming from the humidifier/scrubber which emitted humidified air was introduced into an opening 504 in the sealed BSC 500. A hose 36 coming out of the BSC was attached to the blower 22, thereby providing a closed-loop which circulated humidified air from the humidifier/scrubber 22 into the sealed BSC 500, out of the sealed BSC 500 and back to the humidifier/scrubber 22, and so on.

During the humidification, chlorine dioxide gas was introduced into the BSC as described in Example 1. The BSC was exposed to 4000 ppm chlorine dioxide gas for one hour. In some instances, the air circulation was increased by turning on the BSC fan every 10 minutes for 5 seconds.

Once the hour was up, the chlorine dioxide gas was neutralized as described above. The test strips were cultured as described in Example 1. As shown in Table 2, all spores were killed except on one test strip using the disclosed apparatus and method.

TABLE 2
Decontamination of a BSC using 4000 ppm ClO₂ for 60 min.

| | Ramp Up | Scrub time | Temp | Total Time | |
|---|---|---|---|---|---|
| Run | (min)* | (min)\hat{ } | (° F.) | (min) | Kill# |
| A | 20 | 26 | 80.5 | 106 | 24/24 |
| B | 15 | 20 | 86.0 | 95 | 24/24 |
| C | 15 | 20 | 78.0 | 95 | 24/24 |
| D | 15 | 20 | 76.0 | 95 | 23/24 |
| E | 15 | 20 | 77.0 | 95 | 24/24 |

*amount of time it took to achieve desired humidity and ClO₂ concentration.
\hat{ }amount of time it took to neutralize gas to below 0.1 ppm.
#number of test strips negative for spore growth.

### EXAMPLE 3

### Decontamination of a Biological Safety Cabinet (BSC)

This example describes methods used to decontaminate a BSC containing test strips with pathogenic spores, using the apparatus shown in FIGS. 2 and 3. One skilled in the art will appreciate that similar methods can be used to decontaminate other pieces of laboratory equipment, or entire rooms or buildings.

Test strips described in Example 1 were placed inside a BSC (FIG. 8) in various positions. The cart shown in FIGS. 2 and 3 was wheeled up to the BSC shown in FIG. 8. Referring to FIGS. 2, 3 and 8, the opening in the front of the BSC 500 was sealed using a polyethylene sheet 502 or plastic sheeting and duct tape. An opening 504 was made in the plastic or polyethylene 502 to permit influx of air and other agents through the sealed BSC 500. Gas was evacuated from the sealed BSC using the port 506 on the top of the sealed BSC through fluid flow path 36.

After sealing the BSC, the sealed area was humidified to 70-80% relative humidity as described in Examples 1 and 2. During the humidification, chlorine dioxide gas was introduced into the BSC as described in Example 1. The BSC was exposed to 2500 ppm chlorine dioxide gas for one hour. The BSC circulates 15 s/5 min.

Once the hour was up, the chlorine dioxide gas was neutralized as described above. The test strips were cultured as described in Example 1. As shown in Table 3, all spores were killed using the disclosed apparatus and method.

TABLE 3
Decontamination of a BSC using 2500 ppm ClO₂ for 60 min.

| | Ramp Up | Scrub time | Temp | Total Time | |
|---|---|---|---|---|---|
| Run | (min)* | (min)\hat{ } | (° F.) | (min) | Kill# |
| A | 45 | 10 | 70-74 | 115 | 24/24 |
| B | 48 | 10 | 70-74 | 118 | 24/24 |
| C | 55 | 10 | 70-74 | 125 | 23/23 |
| D | 55 | 15 | 71-74 | 110 | 23/23 |

*amount of time it took to achieve desired humidity and ClO₂ concentration.
\hat{ }amount of time it took to neutralize gas to below 0.1 ppm.
#number of test strips negative for spore growth.

It will be apparent that the precise details of the methods and apparatus described may be varied or modified without departing from the spirit of the described disclosure. We claim all such modifications and variations that fall within the scope and spirit of the claims below.

1. A decontamination method for pathogens, comprising:
providing a sealed environment;
humidifying the sealed environment to at least 70% relative humidity in an absence of negative pressure;
introducing into the sealed environment a concentration of gaseous chlorine dioxide effective to kill substantially 100% of pathogens present in the sealed environment; and
continuously circulating the gaseous chlorine dioxide through the sealed environment using a closed loop circulation system.

2. The method of claim 1, wherein the sealed environment comprises a sealed article, a sealed room, a sealed building, sealed laboratory equipment, or a sealed tissue culture hood.

3. (canceled)

4. (canceled)

5. (canceled)

6. The method of claim 1, wherein humidifying the sealed environment comprises increasing the relative humidity of the sealed environment to no more than 90%, or wherein humidifying the sealed environment to at least 70% relative humidity comprises humidifying the sealed environment for at least one hour.

7. (canceled)

8. The method of claim 1, wherein introducing the concentration of gaseous chlorine dioxide comprises introducing at least 1000 parts per million (ppm) of gaseous chlorine dioxide, or at least 4000 ppm of gaseous chlorine dioxide.

9. (canceled)

10. The method of claim 1, wherein continuously circulating the gaseous chlorine dioxide comprises circulating the gaseous chlorine dioxide through the sealed environment for at least one hour, or for at least one hour but no more than four hours.

11. (canceled)

12. The method of claim 1, wherein the method comprises a method of decontaminating a Bacillus pathogen.

13. The method of claim 1, further comprising neutralizing the gaseous chlorine dioxide in the sealed environment after continuously circulating the gaseous chlorine dioxide gas for a sufficient period of time to kill substantially 100% of the pathogens.

14. The method of claim 13, wherein neutralizing the gaseous chlorine dioxide comprises transporting the gaseous chlorine dioxide in the sealed environment to a solution comprising sodium thiosulfate and a sufficient amount of an inorganic base to maintain alkalinity of the solution during consumption of thiosulfate by the chorine dioxide.

15. The method of claim 14, wherein transporting the gaseous chlorine dioxide in the sealed environment to the solution comprises circulating the gaseous chlorine dioxide between the sealed environment and the solution.

16. The method of claim 14, wherein the inorganic base comprises NaOH.

17. The method of claim 14, wherein the solution comprises 10% by weight each sodium thiosulfate hydrate and NaOH in water.

18. The method of claim 13, wherein the neutralizing proceeds until the concentration of chlorine dioxide reaches less than 0.5 ppm in the sealed environment, or until the concentration of chlorine dioxide reaches less than 0.1 ppm in the sealed environment.

19. (canceled)

20. The method of claim 1, wherein the sealed environment is at ambient temperature and pressure during the humidification and introduction of gaseous chlorine dioxide steps, or during the humidification, introduction of gaseous chlorine dioxide, and neutralization steps.

21. (canceled)

22. The method of claim 1, wherein the sealed environment is at a relative humidity of at least 70% during the introduction of gaseous chlorine dioxide.

23. The method of claim 1, wherein the closed loop circulation system comprises a source of gaseous chlorine dioxide extended to the sealed environment, a gas inlet and a gas outlet that communicate with the sealed environment, and circulating the gaseous chorine dioxide comprises introducing the gaseous chlorine dioxide under pressure into the gas inlet and removing gaseous chlorine dioxide through the gas outlet.

24. The method of claim 23, wherein substantially equal volumes of gaseous chlorine dioxide are introduced into the gas inlet and removed through the gas outlet to promote a substantial steady-state of gaseous chlorine dioxide in the sealed environment.

25. The method of claim 1, further continuously circulating humidified air through the sealed environment during the humidification and introduction of gaseous chlorine dioxide steps using the closed loop circulation system, wherein the closed loop circulation system comprises a source of humidified air extended to the sealed environment, a gas inlet and a gas outlet that communicate with the sealed environment, and circulating the humidified air comprises introducing the humidified air into the gas inlet and removing humidified air through the gas outlet.

26. A decontamination method for pathogens, comprising:
providing a sealed environment;
humidifying the sealed environment to at least 70% but not more than 90% relative humidity in absence of negative pressure;
introducing into the sealed environment at least 2500 ppm gaseous chlorine dioxide for at least one hour but no more than four hours in absence of negative pressure;
neutralizing the gaseous chlorine dioxide in the sealed environment in absence of negative pressure; and
continuously circulating the gaseous chlorine dioxide through the sealed environment using a closed loop circulation system during the introduction, and neutralization steps.

27. A decontamination method for pathogens, comprising:
providing a sealed environment;
humidifying the sealed environment to at least 70% relative humidity in absence of negative pressure;
introducing into the sealed environment a concentration of gaseous chlorine dioxide effective to kill substantially 100% of pathogens present in the sealed environment; and
continuously circulating air in the sealed environment through a humidifier during the humidification and introduction of gaseous chlorine dioxide steps.

28. The method of claim 27, wherein the sealed environment is a sealed room or a sealed building.

29. The method of claim 27, wherein humidifying the sealed environment comprises increasing the relative humidity of the sealed environment to no more than 90%.

30. The method of claim 27, wherein introducing the concentration of gaseous chlorine dioxide comprises introducing 1000 ppm-4000 ppm of gaseous chlorine dioxide.

31. The method of claim 27, wherein continuously circulating the gaseous chlorine dioxide comprises circulating the gaseous chlorine dioxide through the sealed environment for at least one hour, or for at least one hour but no more than four hours.

32. (canceled)

33. The method of claim 27, wherein the method comprises a method of decontaminating a Bacillus.

34. The method of claim 27, further comprising neutralizing the gaseous chlorine dioxide in the sealed environment after continuously circulating the gaseous chlorine dioxide gas for a sufficient period of time to kill substantially 100% of the pathogens.

35. The method of claim 34, wherein neutralizing the gaseous chlorine dioxide comprises transporting the gaseous chlorine dioxide in the sealed environment to a solution comprising sodium thiosulfate and a sufficient amount of an inorganic base to maintain alkalinity of the solution during consumption of thiosulfate by the chorine dioxide.

36. The method of claim 35, wherein transporting the gaseous chlorine dioxide in the sealed environment to the solution comprises circulating the gaseous chlorine dioxide between the sealed environment and the solution.

37. The method of claim 34, wherein the neutralizing proceeds until the concentration of chlorine dioxide reaches less than 0.1 ppm in the sealed environment.

38. The method of claim 27, wherein the sealed environment is at ambient temperature and pressure during the humidification and introduction of gaseous chlorine dioxide steps, or during the humidification, introduction of gaseous chlorine dioxide, and neutralization steps.

39. (canceled)

40. The method of claim 27, wherein the sealed environment is at a relative humidity of at least 70% during the introduction of gaseous chlorine dioxide step.

41. The method of claim 27, wherein the sealed environment is an inhabitable structure, wherein the inhabitable structure comprises a central source of gaseous chlorine dioxide and a fluid flow path from the central source of gaseous chlorine dioxide to one or more rooms in the inhabitable structure, and wherein introducing into the sealed environment a concentration of gaseous chlorine dioxide comprises introducing the gaseous chlorine dioxide through the fluid flow path into the one or more rooms.

42. The method of claim 41, wherein the one or more rooms in the inhabitable structure comprises an air circulation unit, wherein the air circulation unit comprises the humidifier.

43. (canceled)

44. A method of decontaminating an inhabitable structure, comprising:
sealing the inhabitable structure to substantially make the inhabitable structure gas-impermeable, thereby providing a sealed environment;
humidifying the sealed environment to at least 70% relative humidity, but no more than 90% humidity in absence of negative pressure; and
introducing into the sealed environment at least 2500 ppm gaseous chlorine dioxide for at least one hour but no more than four hours in absence of negative pressure;
neutralizing the gaseous chlorine dioxide in the sealed environment in absence of negative pressure, wherein neutralizing comprises exposing gaseous chlorine dioxide in the sealed environment to a solution comprising sodium thiosulfate and NaOH; and
circulating air in the sealed environment through a humidifier during the humidification and introduction of gaseous chlorine dioxide steps, and through a scrubber during the neutralization step, thereby decontaminating the area.

45. (canceled)

46. (canceled)

47. A portable apparatus for decontaminating an environment contaminated with pathogens, comprising:
a movable cart;
a source of chlorine dioxide gas and a humidifier on the cart;
an inlet conduit for introducing a flow of chlorine dioxide gas from the source of chlorine dioxide gas into the environment and for humidifying the environment; and
an outlet conduit for withdrawing gas from the environment and recirculating the gas back to the environment.

48. The portable apparatus of claim 47, further comprising a blower for circulating gas in the environment from the environment to the humidifier and from the humidifier to the environment, a scrubber, a hygrometer for measuring relative humidity in the environment, and/or a device for measuring concentration of chlorine dioxide in the environment.

49. The portable apparatus of claim 47, wherein the inlet conduit comprises a single unit through which the flow of chlorine dioxide gas and humidifier communicate with the environment, or wherein the inlet conduit comprises a first unit through which the flow of chlorine dioxide gas communicates with the environment and a second unit through which humidifier communicates with the environment.

50. (canceled)

51. The portable apparatus of claim 48, wherein the outlet conduit comprises a single unit through which the environment communicates with the blower.

52. (canceled)

53. (canceled)

54. The portable apparatus of claim 48, wherein the scrubber and the humidifier comprise a single unit.

55. (canceled)

56. (canceled)

57. The portable apparatus of claim 47, wherein the apparatus weighs less than 200 pounds.

58. The portable apparatus of claim 47, wherein the apparatus further comprises a diluted chlorine gas in a flow path with the source of chlorine dioxide gas.

59. A method of decontaminating an environment containing or thought to contain a pathogen, comprising sealing the environment, attaching the inlet conduit and outlet conduit of the apparatus of claim 47 to the environment, humidifying the environment, and introducing a flow of chlorine dioxide gas through the environment.

60. The portable apparatus of claim 48, wherein the blower comprises a blower inlet conduit and a blower outlet conduit, thereby establishing a path of flow between blower inlet and blower outlet, wherein blower inlet is in fluid communication with the outlet conduit for withdrawing gas from the environment thereby establishing a path of flow between the environment and the blower, and wherein the blower outlet is in fluid communication with a humidifier inlet conduit on the humidifier thereby establishing a path of flow between the humidifier and the blower.

61. The portable apparatus of claim 48, wherein the humidifier comprises a humidifier inlet conduit and a humidifier outlet conduit, thereby establishing a path of flow between humidifier inlet and humidifier outlet, wherein humidifier inlet is in fluid communication with the blower outlet conduit thereby establishing a path of flow between the humidifier and the blower, and wherein the humidifier outlet is in fluid communication with an inlet conduit for introducing a flow of chlorine dioxide gas into the environment, thereby establishing a path of flow between the humidifier and the environment.

62. The portable apparatus of claim 47, wherein the source of chlorine dioxide gas comprises an outlet conduit that is in fluid communication with the inlet conduit for introducing a flow of chlorine dioxide, thereby establishing a path of flow between source of chlorine dioxide gas and the environment.

63. The portable apparatus of claim 47, wherein the source of chlorine dioxide gas is a chlorine dioxide generator, and the apparatus further comprises a source of diluted chlorine gas comprising a diluted chlorine gas outlet conduit that is in fluid communication with a chlorine dioxide generator inlet conduit on the chlorine dioxide generator, thereby establishing a path of flow between the chlorine dioxide generator and the diluted chlorine gas.

64. The portable apparatus of claim 63, wherein the source diluted chlorine gas comprises 4% chlorine.

65. (canceled)

66. A building with multiple subunits, the building comprising:
a central source of chlorine dioxide gas that communicates with one or more of the multiple subunits through conduits;
a humidifier that provides a source of humidified air; and
a blower that circulates the chlorine dioxide gas and humidified air within one or more selected subunits of the building that is potentially contaminated with a pathogen.

67. The building of claim 66, further comprising a scrubber that provides a source of chlorine dioxide neutralizing agent to the selected subunits following decontamination with the chlorine dioxide gas.

68. The building of claim 67, wherein the scrubber and the humidifier comprise a single unit, or wherein the blower, scrubber and the humidifier comprise a single unit.

69. (canceled)

70. The building of claim 69, wherein the one or more selected subunits comprises the single unit, wherein the unit comprises a first inlet and a first outlet, thereby establishing a path of flow between first inlet and first outlet, wherein first inlet transfers air from the one or more additional subunits into the unit, and wherein the first outlet transfers air from the unit into the one or more additional subunits, thereby permitting circulation in the one or more additional subunits.

71. (canceled)

72. (canceled)

73. The building of claim 66, wherein the source of chlorine dioxide gas is a chlorine dioxide generator, and the room further comprises a source of dilute chlorine gas in fluid communication with the chlorine dioxide generator.

74. The building of claim 66, wherein at least one of the multiple subunits are capable of being sealed so that the subunits are substantially gas-impermeable.

75. (canceled)

76. A building comprising:
a central source of chlorine dioxide gas in fluid communication with one or more of the multiple subunits in the building; and
multiple subunits, wherein at least one of the multiple subunits comprises a humidifier, a blower, and a scrubber, wherein the blower, humidifier and scrubber are a single unit, wherein the unit comprises a first inlet and a first outlet, thereby establishing a path of flow between first inlet and first outlet, wherein first inlet transfers air from at least one of the multiple subunits into the unit, wherein the first outlet transfers air from the unit into the at least one of the multiple subunits, thereby permitting circulation in at least one of the multiple subunits.
